(19) United States
(12) Patent Application Publication
(10) Pub. No.: US 2009/0010859 A1
(43) Pub. Date: Jan. 8, 2009

(54) Oral Composition

(76) Inventor: Philip Christopher Waterfield, Wirral (GB)

Correspondence Address: UNILEVER PATENT GROUP, 800 SYLVAN AVENUE, AG West S. Wing, ENGLEWOOD CLIFFS, NJ, 07632-3100, US

(21) Appl. No.: 11/658,956

(22) PCT Filed: Jul. 13, 2005
(86) PCT No.: PCT/EP05/07722

§ 371 Date: Jan. 30, 2007

(30) Foreign Application Priority Data
Aug. 2, 2004 (EP) 04254662.2
Aug. 3, 2004 (EP) 04254661.4
Nov. 15, 2004 (EP) 04257063.0
Nov. 15, 2004 (EP) 04257064.8
Jan. 13, 2005 (EP) 05250131.9

(51) Int. Cl. A61K 8/365; A61K 8/19; A61Q 11/00; A61K 8/27

(52) U.S. Cl. 424/55; 424/49

(57) ABSTRACT

A visually clear gel toothpaste composition comprising a sparingly soluble divalent metal salt, characterised in that the sparingly soluble divalent metal salt is fully solubilised by a chelating agent for the metal salt, the chelating agent having a log Ks1 for the divalent metal ion as herein defined of from 3.0 to 7.0.

The present invention relates to a visually clear gel toothpaste composition comprising a sparingly soluble divalent metal salt. The invention also relates to a method of making said composition.

EP-A1-0 740 932 (Unilever) discloses a visually-clear gel type dentifrice comprising a zinc salt which is more water soluble than zinc citrate, an amino acid which can bind zinc and a low refractive index type abrasive silica.

In a first aspect of the present invention there is provided a visually clear gel toothpaste composition according to claim 1.

The use of divalent metal salts within toothpaste compositions is well-known in the art as anti-plaque and antimicrobial agents. However, sparingly soluble divalent metal salts are incompatible with visually clear gel toothpastes as they disperse light in such a way as to produce only opaque compositions.

It has surprisingly been discovered that a toothpaste composition according to the present invention can convert a sparingly soluble divalent metal salt into a form compatible for use in a visually clear gel toothpaste. The sparingly soluble divalent metal salt is fully solubilised by a chelating agent for the divalent metal salt, the chelating agent having a log Kₛ1 as herein defined of from 3.0 to 7.0.

In a preferred embodiment of the present invention the sparingly soluble divalent metal is one selected from zinc, tin (II) or copper (II), more preferably the divalent metal is zinc. Suitable zinc salts include zinc citrate, zinc lactate, zinc tartrate, zinc pyrophosphate and zinc maleate. The preferred zinc salt is zinc citrate which, in this type of formulation, provides an optimal balance between making enough zinc ions bioavailable and capable of interacting with bacteria, without compromising the visually clear nature of the toothpaste composition. Preferably, the divalent metal salt is present at from 0.01% to 5.0% by weight of the composition.

The log Kₛ1 is the logarithm of the primary Stability Constant which is the binding affinity for a particular ligand with a particular metal ion. For example, when the chelating agent is citrate and the divalent metal ion is zinc, the primary Stability Constant is the binding affinity for one citrate ligand with the free zinc ion and is defined by the following: Kₛ1=[ML]/[M].[L], where [ML] is the concentration of the metal-ligand complex, [M] is the concentration of the free metal ion and [L] is the concentration of the free ligand. Since [L] is a function of the solution pH, due to the required initial deprotonation of the ligand, as a reference point , the logKₛ1 values described were recorded at pH 7.4.

In a preferred embodiment of the invention the log Kₛ1 of the chelating ligand with the divalent metal ion is equal to or greater than (more preferably greater than) the log Kₛ1 of the acid anion and the divalent metal ion of the sparingly soluble divalent metal salt. Therefore, the additional ligand(s) provided by the chelating agent may be the same as the ligand(s) surrounding divalent metal ion in the sparingly soluble divalent metal salt or the additional ligand(s) may have a greater affinity for the divalent metal ion than the original ligand(s) present. The latter embodiment is preferred in order for the sparingly soluble divalent metal salt to be broken down and fully solubilised in the visually clear gel toothpaste.

The chelating agent is present at from 0.001% to 6% by weight of the composition, preferably at from 0.1% to 4% by weight of the composition. Chelating agents which are suitable for use in the present invention include alkali metal salts of citric acid, acetic acid, tartaric acid, maleic acid and pyrophosphoric acid. The most preferred is citric acid.

Preferably, the excess citrate is incorporated into the composition as an alkali metal citric acid salt such as potassium citrate or sodium citrate. Potassium citrate is most preferable because it provides an optimal level of solubility of the zinc citrate whilst keeping the zinc ions readily bioavailable.

Preferably, the visually clear gel toothpaste composition also comprises abrasive silica. Preferably, the toothpaste composition comprises from 2% to 20% by weight and more preferably, from 5% to 12% by weight abrasive silica. Commercially available abrasive silicas which are suitable for use in the present invention include the Sorbosil AC series supplied by Crosfield, for example Sorbosil AC77, Sorbosil AC39 and Sorbosil AC35 which provide different levels of abrasive cleaning properties. More preferably, silicas with refractive indices ranging from 1.430 to 1.460, most preferably from 1.440 to 1.450, may be used in toothpastes according to the present invention. Sorbosil AC77 is most preferred as it provides excellent cleaning and abrasive properties and is effective at lower levels within the composition enhancing the consumer benefit by reducing the cost.

A useful method for assessing the visual clarity of a toothpaste composition according to the present invention involves the use of a standard chart consisting of black symbols varying in size on a white background. This is the RIT Alphanumeric Resolution Test Object, RT 4-74, produced by Graphic Arts Research Center, Rochester Institute of Technology as described in EP 0 785 169 (Ineos Silicas Limited). The apparatus measures the ability to discern the symbols through a sample of the product of standard thickness (1 cm). The symbols are numbered from −12 to +13. The higher, more positive the number, the greater the clarity. For toothpastes, according to the present invention, a value equal to or greater than 0 is preferable.

The toothpaste composition according to the invention also preferably comprises a fluoride ion source such as an alkali metal salt of fluoride, preferably sodium fluoride. Such fluoride ion source will be present at such an amount to provide free fluoride ion at from 100 to 2000 ppm, preferably from 900 to 1500 ppm.

Preferably, the toothpaste according to the invention comprises an agent selected from the group consisting of anti-caries agents, anti-tartar agents, anti-malodour agents, whitening teeth agents, anti-gingivitis agents and mixtures thereof.

The toothpaste according to the invention comprise further ingredients which are common in the art, such as:

antimicrobial agents, e.g. chlorhexidine, sanguinarine extract, metronidazole, quaternary ammonium compounds, such as cetylpyridinium chloride; bis-guanides, such as chlorhexidine digluconate, hexetidine, octenidine, alexidine; and halogenated bisphenolic compounds, such as 2,2′ methylenebis-(4-chloro-6-bromophenol);

anti-inflammatory agents such as ibuprofen, flurbiprofen, aspirin, indomethacin etc.;

anti-caries agents such as sodium- and stannous fluoride, aminefluorides, sodium monofluorophosphate, sodium trimeta phosphate and casein;

plaque buffers such as urea, calcium lactate, calcium glycerophosphate and strontium polyacrylates;

vitamins such as Vitamins A, C and E;

plant extracts;

desensitising agents, e.g. potassium citrate, potassium chloride, potassium tartrate, potassium bicarbonate, potassium oxalate, potassium nitrate and strontium salts;

anti-calculus agents, e.g. alkali-metal pyrophosphates, hypophosphite-containing polymers, organic phosphonates and phosphocitrates etc.;

biomolecules, e.g. bacteriocins, antibodies, enzymes, etc.;

flavours, e.g. peppermint and spearmint oils;

proteinaceous materials such as collagen;

preservatives;

opacifying agents;

colouring agents;

pH-adjusting agents;

sweetening agents;

pharmaceutically acceptable carriers, e.g. starch, sucrose, water or water/alcohol systems etc.;

surfactants, such as anionic, nonionic, cationic and zwitterionic or amphoteric surfactants;

particulate abrasive materials such as silicas, aluminas, calcium carbonates, dicalciumphosphates, calcium pyrophosphates, hydroxyapatites, trimetaphosphates, insoluble hexametaphosphates and so on, including agglomerated particulate abrasive materials, usually in amounts between 3 and 60% by weight of the oral care composition. The most preferred abrasive is silica.

Humectants such as glycerol, sorbitol, propyleneglycol, xylitol, lactitol etc.;

binders and thickeners such as sodium carboxymethyl-cellulose, hydroxyethyl cellulose (Natrosol®), xanthan gum, gum arabic etc. as well as synthetic polymers such as polyacrylates and carboxyvinyl polymers such as Carbopol®;

polymeric compounds which can enhance the delivery of active ingredients such as antimicrobial agents can also be included;

buffers and salts to buffer the pH and ionic strength of the oral care composition; and

other optional ingredients that may be included are e.g. bleaching agents such as peroxy compounds e.g. potassium peroxydiphosphate, effervescing systems such as sodium bicarbonate/citric acid systems, colour change systems, and so on.

Liposomes may also be used to improve delivery or stability of active ingredients.

In a second aspect of the invention presents a method of making a visually clear gel toothpaste according to the first aspect, the method comprising:
solubilising the divalent metal salt in water;
add the remaining oral care ingredients.

Preferably, the solubilising step comprises mixing in water the sparingly soluble divalent metal salt and an appropriate amount of the chelating agent for the metal ion sufficient to solubilise the divalent metal salt. Complete solubilisation occurs when the solution becomes clear.

Typically visually clear silica gel toothpastes have a pH in the range from 5.5 to 7.0. However, if the visually clear silica gel toothpaste containing the fully solubilised divalent metal salt is required to have a higher pH, for example in the range from 7.0 to 10.0, additional buffering agents may be added. Preferably, sodium hydroxide is employed as a buffering agent in order to raise the pH of the composition to the desired value.

Preferably, humectants and preservatives are added after the divalent metal salt has been fully solubilised and the desired pH reached. Suitable humectants include sorbitol.

Preferably, the silica is added after the humectants and is mixed until the formulation is homogenous. More preferably, the abrasive silica is added together with the foaming agent which is preferably sodium lauryl sulphate. More preferably, these materials are added stepwise to ensure proper mixing.

Preferably, the thickeners are added after the abrasive silica and are mixed until homogenous and air-free.

An embodiment of the invention will now be illustrated by the following non-limiting example.

### EXAMPLE

The following example formulation is an embodiment according to the invention. It is made according to the following steps:

1. Place water, zinc citrate trihydrate, sodium hydroxide and tri-potassium citrate into the mixer and stir until the zinc citrate trihydrate is fully solubilised.

2. Add sorbitol, sweetener, sodium fluoride, colours and polyalkylene glycol to the mixer and stir until fully dissolved.

3. Dry a premix of thickening silica and sodium carboxymethyl cellulose and add to the mixer under vacuum. Stir until homogenous.

4. Dry a premix of abrasives and sodium lauryl sulphate and draw into the mixer under vacuum. Stir until homogenous whilst maintaining the vacuum to remove any entrained air.

5. Add flavour to the mixer and stir to complete the process.

| Ingredient | % w/w |
|---|---|
| Abrasive silica | 8.0 |
| Thickening silica | 8.75 |
| Abrasive silica | 0.5 |
| Sweetener | 0.25 |
| Sodium lauryl sulphate | 2.7 |
| Sodium carboxymethyl cellulose | 0.6 |
| (70% aq) Sorbitol | 65.0 |
| Zinc citrate trihydrate | 0.5 |
| Tri-potassium citrate | 0.5 |
| Sodium hydroxide | 0.015 |
| Colour | 0.0165 |
| Colour | 0.00077 |
| Sodium fluoride | 0.22 |
| Flavour | 1.2 |
| Water, purified | 9.74773 |
| Polyalkylene glycol | 2.0 |

1. A visually clear gel toothpaste composition comprising a sparingly soluble divalent metal salt, characterised in that the sparingly soluble divalent metal salt is fully solubilised by a chelating agent for the metal salt, the chelating agent having a log Kₛ1 for the divalent metal ion as herein defined of from 3.0 to 7.0.

2. A visually clear gel toothpaste composition according to claim 1, wherein the log Kₛ1 of the chelating agent with the divalent metal ion is equal to or greater than the log Kₛ1 of the acid anion and the divalent metal ion of the sparingly soluble divalent metal salt.

3. A visually clear gel toothpaste composition according to claim 1, wherein the sparingly soluble divalent metal salt is selected from zinc, tin (II) or copper (II) salts.

4. A visually clear gel toothpaste composition according to claim 1, wherein the sparingly soluble divalent metal salt is a zinc salt.

5. A visually clear gel toothpaste composition according to claim 1, wherein the sparingly soluble divalent metal salt is zinc citrate.

6. A visually clear gel toothpaste composition according to claim 1, wherein the chelating agent is an alkali metal salt of an acid selected from citric acid, acetic acid, tartaric acid or pyrophosphoric acid.

7. A visually clear gel toothpaste composition according to claim 1, wherein the chelating agent is an alkali metal salt of citric acid.

8. A visually clear gel toothpaste composition according to claim 1, wherein the alkali metal is selected from sodium or potassium.

9. A visually clear gel toothpaste composition according to claim 1, wherein the alkali metal salt of citric acid is potassium citrate.

10. A visually clear gel toothpaste composition according to claim 1, wherein the divalent metal salt is present at from 0.01% to 5.0% by weight of the composition.

11. A visually clear gel toothpaste composition according to claim 1 comprising an abrasive silica.

12. A visually clear gel toothpaste composition according to claim 1 comprising an agent selected from the group consisting of anti-caries agents, anti-tartar agents, anti-malodour agents, whitening teeth agents, anti-gingivitis agents and mixtures thereof.

13. A method for making a toothpaste according to claim 1, comprising mixing in water a sparingly soluble divalent metal salt with a chelating agent having a log Ks1 for the divalent metal ion of from 3.0 to 7.0 until the divalent metal salt is fully solubilised and then adding the remaining oral care ingredients.
